(19) United States
(12) Patent Application Publication
(10) Pub. No.: US 2015/0283080 A1
(43) Pub. Date: Oct. 8, 2015

(54) STABILIZED DERMATOLOGICAL DELIVERY SYSTEM FOR ACTIVE INGREDIENT COMPOSITIONS FOR TOPICAL ADMINISTRATION TO THE SKIN

(71) Applicant: PANKAJ MODI, Ancaster (CA)

(72) Inventor: PANKAJ MODI, Ancaster (CA)

(21) Appl. No.: 14/243,955

(22) Filed: Apr. 3, 2014

(51) Int. Cl. A61K 9/16; A61K 9/00; A61K 31/728; A61Q 19/08; A61K 8/02; A61K 8/73

(52) U.S. Cl. CPC A61K 9/16; A61K 8/025; A61K 8/735; A61K 31/728; A61Q 19/08; A61K 9/0014; A61K 2800/412

(57) ABSTRACT

The invention relates to a stabilized dermatological delivery system for active ingredient compositions for topical administration to the skin, one or more micelle forming compounds, one or more skin penetration enhancers, a surfactant, and one or more solvents, wherein the active ingredient is solubilized in the solvent. The invention further relates to the process for making the topical formulation.

### RELATED APPLICATIONS

THIS APPLICATION CLAIMS THE PRIORITY OF AND IS A CONTINUATION IN PART OF U.S. patent application Ser. No. 12/133,939 FILED Jun. 5, 2008, AND U.S. patent application Ser. No. 12/126,594 FILED May 23, 2008, AND IS ALSO A CONTINUATION IN PART OF U.S. patent application Ser. No. 11/259,778 FILED Oct. 27, 2005, NOW U.S. Pat. No. 7,727,537, WHICH IS A CONTINUATION IN PART OF U.S. patent application Ser. No. 11/057,481, FILED Feb. 14, 2005, NOW U.S. Pat. No. 7,838,011, THE ENTIRE CONTENTS AND DISCLOSURE OF EACH AND ALL OF THEM IS EXPRESSLY INCORPORATED HEREIN BY REFERENCE AS IF FULLY SET FORTH.

### FIELD

This patent application relates to a method for stabilizing active ingredients in pharmaceutical compositions, producing cross-linked hyaluronic acid microbeads, providing prolonged and enhanced shelf-live at room temperatures to the active ingredients and delivering those active ingredients to effected areas by topical application and transdermal delivery.

### BACKGROUND

An active agent that would be desirable to deliver through the skin is hyaluronic acid (HA). Hyaluronic acid is a naturally occurring high molecular weight polysaccharide that is found in many tissues of the body. Hyaluronic acid has been associated with maintaining moisture in the skin as well as with promoting wound healing and encouraging the formation of vessels. German Patent DE 19805847A describes the protective effect of hyaluronic acid on skin irritations. U.S. Pat. No. 5,728,391 also suggests the use of hyaluronic acid as an agent for treating skin disease.

Various formulations for the oral delivery of HA have been suggested. To enhance the effect of HA on the skin, it is desirable to formulate a composition that can be applied topically to the skin. One of the difficulties, however, in trying to increase the permeation of HA in the skin is the size of the molecule. The large polymeric structure that gives HA its beneficial effects also makes it difficult to acquire from outside the body. Thus, there was an unmet need for improved formulations of HA that can be applied topically.

The process that leads to skin aging and wrinkles is complex. A primary cause of wrinkling is a build-up of free radical toxic plaque that binds to collagen and elastin fibers, causing the skin's supportive structure to become inflexible and unhealthy. Laugh lines, smile lines, crow's feet or facial creases appear in areas where repeated muscle movement occurs. Thus, it would be desirable to be able to deliver free radical scavengers to the skin.

The aging skin of older people differs from the normal skin of younger people in a plurality of symptoms. It is generally drier and shows an uneven hornification. Through its lack of water-binding capacity in the corium, deep wrinkles develop. The tendency of the epidermis to form vesicular flaking is increased. The appearance of old skin develops in genetic aging as with chronic environmental damage, as is caused, e.g., by excessive UV exposure. Exogenous factors, such as UV light and chemical noxae, can have a cumulative effect and, e.g., accelerate or supplement the endogenous aging processes. In the epidermis and dermis, for example, the following structural damage and functional disorders appear in the skin as a result of exogenous factors:

a) Visible telangiectasis (cuperosis);
b) Flaccidity and the development of wrinkles;
c) Local hyperpigmentation, hypopigmentation and defective pigmentation (e.g., age spots);
d) Increased susceptibility to mechanical stress (e.g., cracking);
e) Decrease in the collagen content of the skin (e.g., through reduced new synthesis and/or through increased decomposition)
f) Disturbances in the glycosaminoglycan and elastin metabolism.

Botulinum toxin, hyaluronic acid and anti-oxidants are just a few examples of active agents that it is desirable to deliver to the skin and which may be used in connection with this invention. Other active agents may include enzyme inhibitors, vasodilators, perflourocarbons, hormones, growth factors, vaccines, drugs, small molecules, amines, peroxides, analgesics and other therapeutic agents. Agents which promote wound healing or reduce pain are also active agents that it would be desirable to administer through the skin.

Nowadays, consumers are offered a large number of cosmetic preparations for skin care, generally in the form of creams and lotions, i.e., as an emulsion. Products that temporarily or permanently delay or remove signs of aging in the skin (in particular the development of fine lines and wrinkles) thereby have steadily increasing importance. In addition to water for moisturizing the skin and oils and lipids for regreasing the skin, skin care products of this type contain a plurality of active substances, auxiliaries and additives.

Conventional skin care products for the prophylaxis and treatment of skin aging symptoms, however, have the disadvantage that these active substances as a rule can be incorporated into cosmetic formulations only with difficulty and in unsatisfactory amounts. Furthermore, according to the prior art the disadvantage regularly occurs that combinations of active substances are difficult to incorporate into the preparations, since the active substances can exhibit incompatibility not only with the carrier preparation but also among one another.

Thus, there was a need for newer methods for stabilization of active agents for the preparation of topical and cosmetic preparation. There was also a need for improved systems for delivery of active agents to the skin. The present invention addresses those needs for both large and small molecules, including cross-linked HA and Botulinum toxin, among others.

Relatively little progress has been made in reaching the target of safe and effective non-invasive transdermal delivery of formulations for macromolecules, including peptides and proteins. Barriers to developing transdermal formulations for proteins, peptides and other large and small molecules include poor intrinsic permeability, cellular enzymatic degradation and chemical instability. Pharmaceutical approaches to address these barriers that have been successful with traditional small, organic drug molecules have not readily translated into effective peptide and protein formulations. The ability of molecules to permeate the skin effectively appears to be related to molecular size, lipid solubility and peptide protein ionization. Molecules less than 1000 daltons appear to cross the skin barriers rapidly. As molecular size increases, the permeability of the molecule decreases rapidly. Lipid soluble compounds arc more permeable than non-lipid soluble molecules. Maximum absorption occurs when molecules are un-ionized or neutral in electrical charges. Charged molecules, therefore, present the biggest challenges to absorption through the skin.

Some enhancers, especially those related to bile salts, and some protein solubilizing agents are extremely potent in transporting the molecules effectively across the tight junctions and skin. Several approaches have been utilized to improve the transport of the bile salt-based delivery systems, including the use of protease inhibitors and various polymer matrices. Other attempts to deliver large molecules using single bile acids or enhancing agents in combination with protease inhibitors and biodegradable polymeric materials similarly failed to achieve therapeutic levels of proteinic drugs in the patient. Single enhancing agents fail to loosen tight cellular junctions for the time needed to permit passage of large molecules through the skin membranes without further degradation.

Various transmission systems have been proposed in connection with the delivery of small molecules such as local anesthetic compounds. U.S. Pat. No. 5,013,545 to Blackmon et. al. discloses aqueous gel-containing topical medications comprising high concentrations of alcohol, water and topically effective amounts of a pharmaceutical active such as hydrocortisone, diphenhydramine hydrochloride, lidocaine or miconazale nitrate in a gel matrix primarily consisting of water-soluble carboxyvinyl polymers. A gel clarifying agent may be optionally added for aesthetic reasons.

U.S. Pat. No. 4,937,078 to Mezie et. al. discloses the incorporation of certain concentrations of topical anesthetic actives into liposomes which arc of a substantially greater size than nano particles. U.S. Pat. No. 5,081,158 to Pomerantz discloses the use of medicated protective films as a carrier for topical anesthetics. The films are comprised of hydroxypropyl cellulose (HPC) and an esterification agent which renders the HPC soluble in a non-volatile solvent such as ethanol, isopropanol or methanol. Medicinal compounds such as benzocaine and a variety of other topical anesthetics, antibiotics and steroids are incorporated which, when applied to the skin, result in situ formed medicated films from which the actives are released to provide a sustained supply of the medicine at the treatment site.

U.S. Pat. No. 5,002,974 to Geria discloses a topical anesthetic and skin moisturizing composition comprising any one of a number of topical anesthetics, including pramoxine, in an oil-in-water emulsion including a dissolved surface active agent. The composition is asserted to provide an aesthetically pleasing analgesic skin care product. The emulsion not only provides relief from the pain associated with irritated skin but is asserted to soften and moisturize the skin with an oily coating. U.S. Pat. No. 4,493,591 to Fourman et al discloses skin care cosmetic formulations comprised of a cellulosic polymer/solvent system capable of dispersing thin, substantive films upon the skin. Such films may serve as a carrier for sun blocking agents and insect repellents and also serve to prevent water loss form the skin surface to the environment.

Finally, U.S. Pat. No. 4,389,418 to Burton et. al., in a more general and traditional sense, discloses the use of hydrocarbons such as petrolatum, paraffin wax and ozokerite and other emollients as skin moisturizing materials. These function by covering the skin with a hydrophobic occlusive film which prevents water loss from the skin to the environment.

Hyaluronic acid and preparations thereof have been known for a long time. Hyaluronic acid is an important constituent of human tissue and is present in the human body in the eye (vitreous humor), in bone joints and the epidermis. There arc ca. 15 g of hyaluronic acid in the body of a person weighing 70 kg.

Hyaluronic acid is a macromolecular chain of disaccharides which consists of two glucose derivatives: D-glucuronic acid and N-acetyl-D-glucosamine. In the disaccharide, the glucuronic acid is linked to the N-acetylglucosamine, which in turn is f1(1?4) joined to the next glucuronic acid in the polymeric chain. A chain here typically consists of 250-250 000 disaccharide units.

Hyaluronic acid and its preparations are used in the cosmetics sector, the pharmaceutical sector, and also the medical sector. In medicine, hyaluronic acid preparations are used for the treatment of joint complaints, in particular arthritic complaints. Here, the hyaluronic acid serves, injected directly into the joint, as joint lubricant, for improving the mobility of the joint apparatus. A further possible use of hyaluronic acid preparations in medicine is the field of ophthalmology, in particular cataract surgery. In the large field of aesthetics and cosmetics, hyaluronic acid is used in particular as a water store. Both in preparations to be used topically, and also in the case of the injection. By injecting hyaluronic acid directly under the skin, water depots are thus formed which expand like a sponge, and visibly decrease wrinkles on the outside, and when injected into lips, make these look full and well-shaped. Applied topically, hyaluronic acid increases the water retention capacity of the skin, as a result of which the transepidermal water loss (TEWL) is considerably reduced. The skin retains a fresh, youthful and tightened appearance and at the same time remains elastic and supple. Moreover, upon multiple application also in the case of hyaluronic acid preparations applied topically, small lines are reduced.

### SUMMARY

In one aspect, the present invention discloses a solubilized topical formulation that comprises either a small or large molecule agent, one or more micelle forming compounds, one or more solvents, one or more skin penetration enhancers, and a surfactant.

In another aspect, the present invention discloses and relates to methods of producing crosslinked hyaluronic acid microbeads, as well as the produced microbeads, where the method is comprised of the steps of: (a) providing an aqueous alkaline emulsion, suspension or solution comprising hyaluronic acid, or a salt thereof; (b) forming microdroplets having a desired size from the mixed solution of step (a) in an organic or oil phase to form a water in organic or water in oil (W/O) emulsion, wherein the amount of oil phase used is of from 20 to less than 50% by weight based on the sum of oil phase and water.

In yet another aspect, it is an object of the present invention to develop a new delivery systems consisting of nano-particles, micelles, stable and cosmetically effective skin care preparation for the prophylaxis and treatment of skin aging manifestations, in particular fine lines and wrinkles.

Many of the small molecule actives are not completely soluble in water alone or mixture of water and alcohols at room temperature. Accordingly, the small molecule actives are solubilized completely with either short chain or long chain alcohols.

The formulation includes a volatile, short-chain alcohol such as isopropyl alcohol or ethanol or isomers or butanol etc to effect a complete solubilization. The short-chain alcohol forms between 20 to 50 percent by weight of the formulation.

The solubilized small molecule active is then combined and agitated to form a mixed micelles aggregation comprise of both nano particle (1-10 nm size) and some larger particles (from 100-nm or larger). The micelles are mixed micelles formed between the solubilized small molecule active in the mixture of alcohols and water and SDS, Tweens, bile acids, salicylate, and glycerol, QTS, squaimsom (ATS), EDTA, Polysorbate (tween)-20 and other micelles forming agents such as lipid molecules, hyaluronic acid, oong chain alcohols, lanolin, Hohoba oil.

To enhance the penetration the micelles may be further coated with lecithin, (in either saturated or unsaturated form), or phosphatidylcholine, and lysolecithin and mixtures thereof. The delivery system's effectiveness may be further enhanced by combining the mixed micelles of small molecule actives with slicylate, lactic acids, triolein, polyoxyethylene ethers or polidocanol alkyl ethers. The nano particle drug delivery system for topical administration and transdermal delivery of either a small or large molecule agent is comprised of: a small molecule agent;

a. a micelle for encapsulating the small molecule agent; and,

b. a lipid molecule layer coating substantially all of each micelle

Various small or large molecule actives may be substituted.

The small molecules are solubilized in alcohol mixture with water inasmuch as many of the small molecule agents are not very soluble in water. The small molecules do not require further stabilization by coating them with other lipids or protein molecules to store them at a room temperature for a long time period (months to year).

The present invention also relates to product produced by crosslinked hyaluronic acid microbeads, as well as the produced microbeads, where the product is produced by a method comprising the steps of: (a) providing an aqueous alkaline emulsion, suspension or solution comprising hyaluronic acid, or a salt thereof; (b) forming microdroplets having a desired size from the mixed solution of step (a) in an organic or oil phase to form a water in organic or water in oil (VWO) emulsion, wherein the amount of oil phase used is of from 20 to less than 50% by weight based on the sum of oil phase and water.

An object of the present invention also is to develop microbeads that can be deployed via a new delivery systems consisting of nano-particles, micelles, stable and cosmetically effective skin care preparation for the prophylaxis and treatment of skin aging manifestations, in particular fine lines and wrinkles.

The Microbead Molecule Delivery System (“MMDS”) described above fulfills the need by providing an improved delivery of pharmaceutical compositions comprising a macromolecular pharmaceutical agent, an alkali metal alkyl sulfate, a pharmaceutically acceptable edetate, an alkali metal salicylate and at least one additional micelle-forming compound, in a suitable solvent. The agent can be one or more proteins, peptides, hormones, vaccines or drugs. The molecular weight of the macromolecular pharmaceutical agent preferably ranges between about 1,000 and 2,000,000 daltons. The agent is presented in micellar form, with a micelle size of approximately one to 10 nanometers (nm).

As used herein the term “mixed micelles” refers to at least two different types of micelles, each of which has been formed using different micelle forming compounds: for example, the present compositions comprise a mix of at least two different types of micelles—micelles formed between the pharmaceutical agent and alkali metal alkyl sulfate, and micelles formed between the pharmaceutical agent and at least one different additional micelle forming compound as disclosed herein. It will be understood that each individual micelle can be formed from more than one micelle forming compound as well. The mixed micelles of the present invention tend to be smaller than the pores of the membranes (skin). It is therefore believed that the extremely small size of the present mixed micelles helps the encapsulated macromolecules penetrate efficiently through the skin. Thus, the present compositions offer increased bioavailability of active drug, particularly across the skin, when compared with pharmaceutical preparations known in the art. The MMDS delivery system also enhances the rate of absorption of macromolecular pharmaceutical agents, such as cross-linked HA, comprising the agent in combination with an alkali metal alkyl sulfate, a pharmaceutically acceptable edetate, at least one alkali metal salicylate, and at least one micelle-forming compound.

The MMDS delivery system is a pharmaceutical composition comprising of an effective amount of a macromolecular pharmaceutical agent; an alkali metal alkyl sulfate; a pharmaceutically acceptable edetate; at least one alkali metal salicylate; at least one micelle-forming compound selected from the group consisting of lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysinc, triolein, polyoxyethylene ethers, polidocanol alkyl ethers, chenodeoxycholate, deoxycholate, pharmaceutically acceptable salts thereof, analogs thereof and mixtures or combinations thereof, and a suitable solvent. The alkali metal alkyl sulfate, the edetate, and the alkali metal salicylate are each present in a concentration between about 1 and 20 wt./wt. % of the total composition, each micelle-forming compound concentration is between about 1 and 20 wt./wt. % of the total composition, and the total concentration of the alkali metal alkyl sulfate, edetate and the micelle-forming compound together is less than 50 wt./wt. % of the total composition.

The solubilized topical formulation may comprise one or more solvents that are selected from ethylene glycol-400 (low molecular weight solvent), propylene or butylene glycol, isopropyl or ethyl alcohol, short chain alkyl esters, or combinations thereof.

The solubilized topical formulation may comprise the surfactant sodium lauryl sulfate. The solubilized small molecule agent topical formulation may comprise the skin penetration enhancer dimethylsulfoxide (DMSO).

In a preferred embodiment, the solubilized topical formulation comprises: small molecule agent, the skin penetration enhancer dimethylsulfoxide (DMSO), the surfactant sodium lauryl sulfate and the solvents: ethylene glycol-400, propylene or butylene glycol and isopropyl or ethyl alcohol.

In one aspect, this patent application discloses a stabilized topically administered botulinum toxin composition for the skin of a patient, wherein the botulinum toxin composition comprises an effective amount of botulinum toxin encapsulated in phospholipid micelles, one or more primary stabilizers, and one or more skin penetration enhancers.

In another aspect, this patent application discloses a stabilized topically administered cross-linked HA composition for the skin of a patient, wherein the HA composition comprises and effective amount of cross-linked HA encapsulated in phospholipid micelles, one or more primary stabilizers, and one or more skin penetration enhancers.

In another embodiment, this patent application discloses the use of the solubilized topical formulation for the treatment and/or prophylaxis of acne.

In another embodiment, this patent application discloses the use of the solubilized topical formulation for the application and transdermal delivery of antibiotics.

In another embodiment, this patent application discloses the use of the solubilized topical formulation for the application and transdermal delivery of antimicrobials.

In another embodiment, this patent application discloses the use of the solubilized topical formulation for the application and transdermal delivery bactericidals.

In a further embodiment, this patent application discloses a process for the preparation of the solubilized topical formulation.

The solubilized topical formulation may include additional ingredients to form an emulsion, suspension, cream, lotion, get or stick for topical administration. The phospholipid micelles may comprise sphingosine and cerebroside, for example. The primary stabilizers may comprise elastin and collagen, for example. One or more of the skin penetration enhancers may be selected from the group that includes d-limonene, allantoin, fulvic acid, myrrh, hydroquinone glyquin, quillaja saponaria (QTS), and acanthophyllum squarrusom (ATS).

### BRIEF DESCRIPTION OF THE DRAWINGS

Embodiments of the invention will be described, by way of example only, with reference to the appended drawings, wherein:

FIG. 1 is a chart setting forth a clinical evaluation of skin roughness for participants in a double blind vehicle controlled trial.

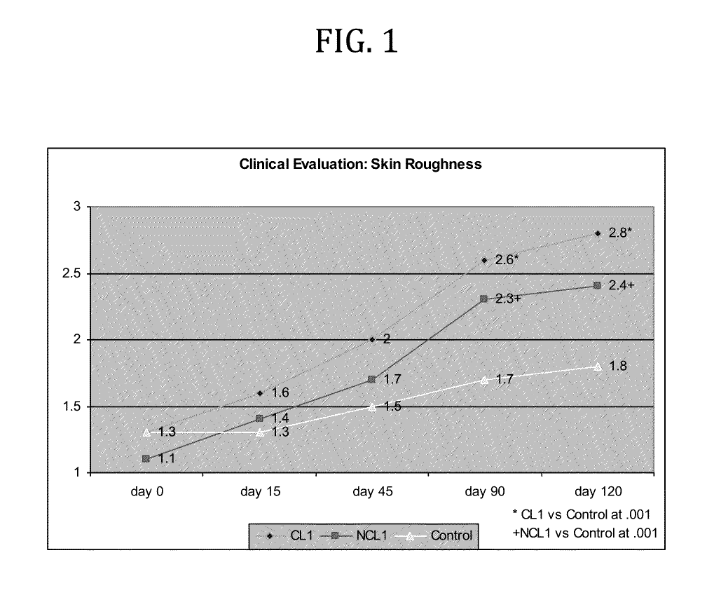

FIG. 2 is a chart setting forth a clinical evaluation of skin hydration for participants in a double blind vehicle controlled trial.

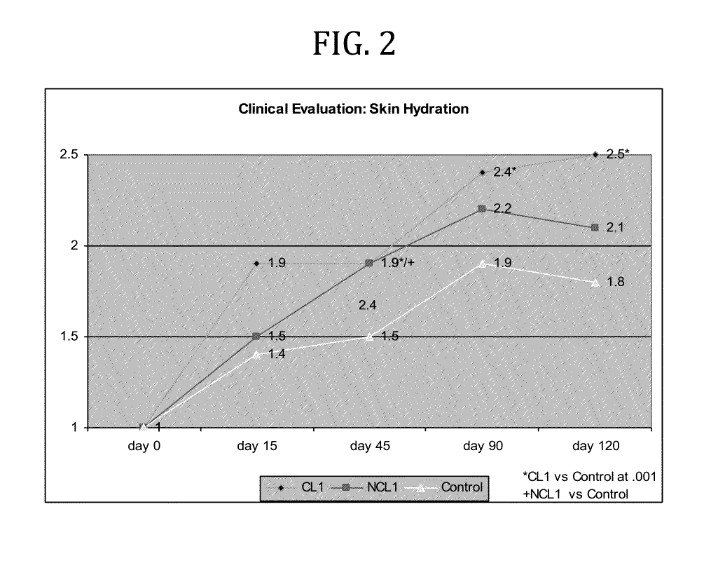

FIG. 3 is a chart setting forth a clinical evaluation of skin elasticity for participants in a double blind vehicle controlled trial.

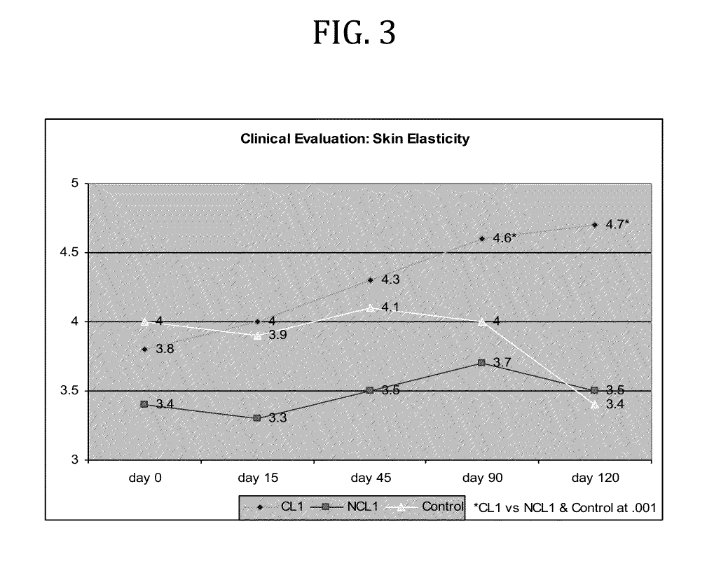

FIG. 4 is a chart setting forth a clinical evaluation of skin radiance for participants in a double blind vehicle controlled trial.

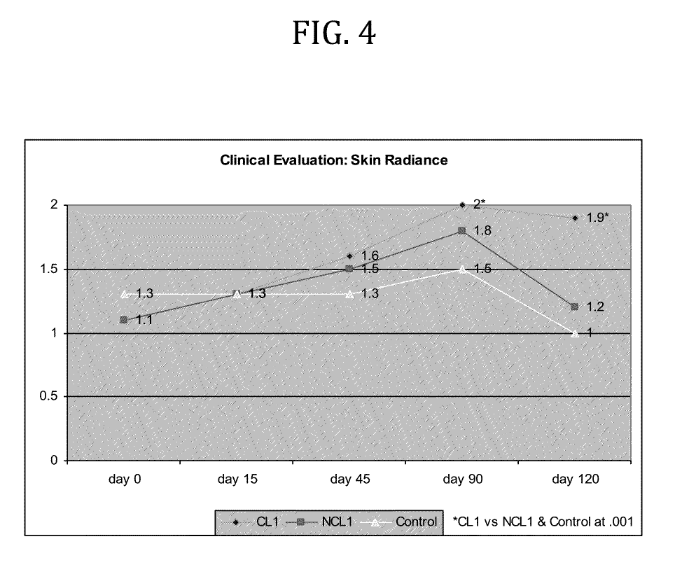

FIG. 5 is a chart setting forth a clinical evaluation of skin smoothing effect for participants in a double blind vehicle controlled trial.

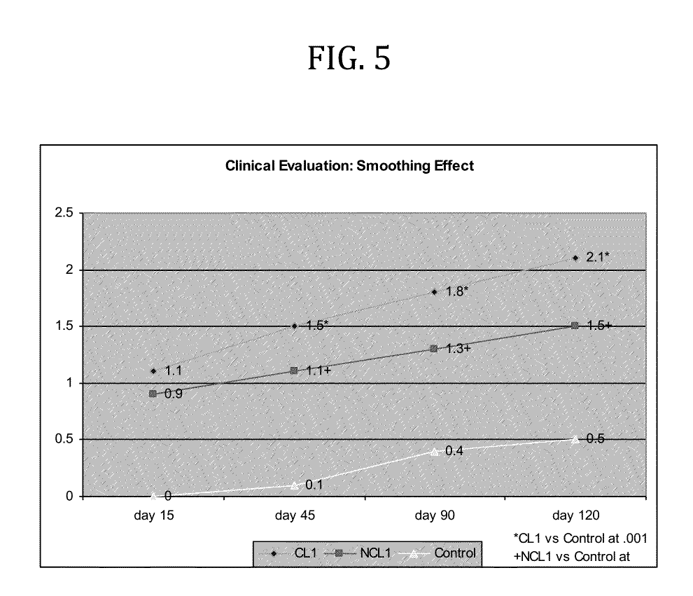

FIG. 6 is a chart setting forth a clinical evaluation of oval efficacy for participants in a double blind vehicle controlled trial.

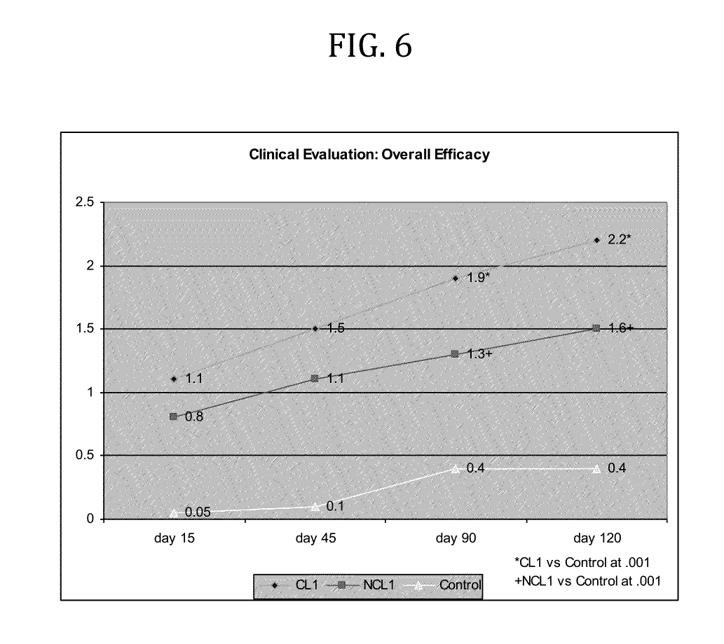

### DETAILED DESCRIPTION

Methods of preparing a pharmaceutical or cosmetic composition for topical delivery of at least one active agent (such as botulinum toxin or cross-linked HA), and compositions prepared by these methods, will now be described.

The composition comprises an effective amount of an active ingredient encapsulated in phospholipid micelles, one or more primary stabilizers, and one or more skin penetration enhancers. The composition may be formulated in any form suitable for topical or transdermal administration.

### A Method of Preparation of Composition—Overview

The topical composition is prepared by encapsulating an active ingredient in a phospholipid micelle. The micelle solution is then preferably combined with a base composition that includes one or more primary stabilizers, such as collagen and elastin.

Briefly, the phospholipid is dissolved in a suitable solvent, such as an alcohol. For example, the phospholipid may be dissolved in ethanol or a mixture of ethanol and isopropanol. The alcohol is removed by, for example, rotary vacuum evaporation. An aqueous solution containing the active ingredient is then added. The active ingredient thus becomes encapsulated by a phospholipid micelle structure. This solution can then be combined with a base solution comprising collagen and elastin. In describing and claiming the present invention, the following terminology will be used in accordance with the definitions set forth below.

The singular forms “a,” “an,” and “the” include plural referents unless the context clearly dictates otherwise. Thus, for example, reference to “a solvent” includes reference to one or more of such solvents, and reference to “the dispersant” includes reference to one or more of such dispersants.

As used herein, “formulation” and “composition” may be used interchangeably and refer to a combination of elements that is presented together for a given purpose. Such terms are well known to those of ordinary skill in the art.

As used herein, “carrier,” “inert carrier,” and “acceptable carrier” may be used interchangeably and refer to a carrier which may be combined with a one or a plurality of agents in order to provide a desired composition. Those of ordinary skill in the art will recognize a number of carriers that are well known for making specific remedial compositions.

As used herein, “biologically acceptable carrier” refers to a material which is suitable for use in connection with a particular biological material. A biologically acceptable carrier is compatible with, and does not adversely affect, a biological material or subject contacted therewith under prescribed conditions.

As used herein, “cosmetic” is an adjective referring to improving the appearance of a surface or covering defects. Typically, cosmetic compositions can be used to improve aesthetic rather than functional aspects of a surface. Most commonly, cosmetic compositions are formulated for application as a beauty treatment or for affecting personal appearance of the body, for example, natural tooth enamel and dental veneer surfaces.

As used herein, “remedial” is an adjective referring to remedying, correcting, treating, improving, or preventing an undesirable condition. A remedial composition can therefore be formulated to remove undesirable stains from the surface of natural tooth enamel or veneer. Similarly, remedial compositions can be configured to remove, prevent or minimize formation of undesirable elements such as stain build up and the like.

As used herein, “biological material” refers to any material which is a product of a biological organism. Typical biological materials of interest can include organic oils and the like.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of 1 to 5 should be interpreted to include not only the explicitly recited limits of 1 and 5, but also to include individual values such as 2, 2.7, 3.6, 4.2, and sub-ranges such as 1-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10”.

The term “volatile component” as used herein refers to a component (e.g., a solvent or combination of solvents) that changes readily from solid or liquid to a vapor, e.g., that evaporates readily at some temperature at or below body temperature and less readily at room temperature, such as a component that evaporates rapidly between 21 and 37.degree. C. at atmospheric pressure.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “saturation” refers to the point at which a solution of a substance (e.g., a local anesthetic agent) can dissolve no more of that substance and additional amounts of it will appear as a precipitate. The phrase “near saturation” refers to a solution which is at least 90% saturated, such as 90%, 91%, 92%, 93%, 94%, 95%, 96%, 97%, 98%, or 99% saturated. The phrase “above saturation” refers to a solution which has a higher concentration of substance (e.g., a local anesthetic agent) than the concentration at which the solution is saturated (e.g., it is greater than 100% saturated).

The drug delivery system and methods of the present invention may be utilized to treat an individual in need thereof. In certain embodiments, the individual is a mammal such as a human, or a non-human mammal

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The formulations of the present invention can be administered to a subject topically, for example, as a gel, foam, solution, lotion, cream, ointment or spray applied to the skin.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the anesthetic agent which produces an anesthetic effect.

The formulations of the present invention for topical or transdermal administration include powders, sprays, ointments, pastes, creams, lotions, gels, solutions, patches and inhalants. The anesthetic agent may be mixed under sterile conditions with the other components of the drug delivery system, and with any preservatives, buffers, or propellants that may be required.

The formulations of the present invention may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like.

Actual dosage levels of the active ingredients in the formulations may be varied so as to obtain an amount of the active ingredient that is effective to achieve the desired anesthetic response for a particular patient, composition, and mode of administration, without being toxic to the patient.

The selected dosage level will depend upon a variety of factors including the activity of the particular agent or combination of agents employed, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the formulation required. For example, the physician or veterinarian could start doses of the formulations at levels lower than that required in order to achieve the desired anesthetic effect and gradually increase the dosage until the desired effect is achieved. By “therapeutically effective amount” is meant the concentration of the agent that is sufficient to elicit the desired effect. It is generally understood that the effective amount of the agent will vary according to the weight, sex, age, and medical history of the subject.

Other factors that influence the effective amount may include, but are not limited to, the severity of the patient's condition, the disorder being treated, the stability of the agent of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

As used herein, the term “macromolecular”, when used in conjunction with the term pharmaceutical agent, refers to pharmaceutical agents having a molecular weight greater than about 1000 daltons; preferably the macromolecular pharmaceutical agents of the present invention have a molecular weight between about 1000 and 2,000,000 daltons although even larger molecules are also contemplated.

The macromolecular pharmaceutical agent exists in micellar form in its intact pharmaceutical composition. A micelle is a colloidal aggregate of amphipathic molecules in which the polar hydrophilic portion of the molecules extends outwardly while the non-polar hydrophobic portion extends inwardly. The micelle encapsulates the molecule of interest. As discussed below, various combinations of micelle-forming compounds are utilized in order to achieve the present formulation. It is believed that the presence of the micelles significantly aids in the absorption of the macromolecular pharmaceutical agent both because of their enhanced absorption ability, and also because of their size. The particle size of the micelles will typically be in the range of 1 to 10 nanometers. Preferably, the micelle size ranges between 1 and 5 nanometers.

The following definitions apply herein: “About” means approximately or nearly and in the context of a numerical value or range set forth herein means 10% of the numerical value or range recited or claimed.

“Local administration” means direct administration of a pharmaceutical at or to the vicinity of a site on or within an animal body, at which site a biological effect of the pharmaceutical is desired. Local administration excludes systemic routes of administration, such as intravenous or oral administration. Topical administration is a type of local administration in which a pharmaceutical agent is applied to a person's skin. Topical administration of a neurotoxin, such as botulinum toxin, excludes systemic administration of the neurotoxin. In other words, and unlike conventional therapeutic transdermal methods, topical administration of botulinum toxin does not result in significant amounts, such as the majority of, the neurotoxin passing into the circulatory system of the patient.

“Enhancing agent” refers to an agent that enhances the permeability of a patient's skin so that the benzoyl peroxide can be absorbed by the skin to achieve a therapeutic effect. In reference to the disclosure herein, enhancing agent specifically includes dimethylsulfoxide (DMSO) or a combination of pluronic lecithin organizer (PLO) and DMSO. An enhancing agent may include, and is not limited to, alcohols, such as short chain alcohols, long chain alcohols, or polyalcohols; amines and amides, such as urea, amino acids or their esters, amides, AZONE®, derivatives of AZONE®, pyrrolidones, or derivatives of pyrrolidones; terpenes and derivatives of terpenes; fatty acids and their esters; macrocyclic compounds; tensides; or sulfoxides other than dimethylsulfoxide, such as, decylmethylsulfoxide; liposomes; transfersomes; lecithin vesicles; ethosomes; water; surfactants, such as anionic, cationic, and nonionic surfactants; polyols; and essential oils.

Substances that facilitate the absorption or transport of large molecules (>1000 daltons) across biological membranes are referred to in the art as “enhancers” or “absorption aids.” These compounds include chelators, bile salts, fatty acids, synthetic hydrophilic and hydrophobic compounds, and biodegradable polymeric compounds. Many enhancers lack a satisfactory safety profile respecting irritation, lowering of the barrier function, and impairment of the mucocilliary clearance protective mechanism.

The compositions of the MMDS delivery system further comprise at least one micelle-forming compound selected from the group comprising lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglycerin, lysine, polylysine, triolein, polyoxyethylene ethers, polidocanol alkyl ethers, chenodeoxycholate, deoxycholate, pharmaceutically acceptable salts thereof, analogs thereof and mixtures or combinations thereof.

Each micelle-forming compound listed above is present in the composition in a concentration of between about 1 and 20 wt./wt. % of the total composition. More preferably, each micelle-forming compound is present in a concentration of between about 1 and 5 wt./wt. % of the total composition. The alkali metal alkyl sulfate functions as a micelle forming agent, and is added to the composition in addition to the one or more other micelle-forming compounds listed herein. The total concentration of alkali metal alkyl sulfate, the edetate and the micelle-forming compounds together is less than 50 wt./wt. % of the total composition.

The lecithin can be saturated or unsaturated, and is preferably selected from the group consisting of phosphatidylcholine, phosphatidylserine, sphingomyelin, phosphatidylethanolamine, cephalin, and lysolecithin and mixtures thereof. Saturated and unsaturated lecithin are commercially available from. The American Lecithin Co. as Phospholipon-H™ and Phospholipon-G™, respectively.

Preferred salts of hyaluronic acid are alkali metal hyaluronates, especially sodium hyaluronate, alkaline earth hyaluronates, and aluminum hyaluronate. When using hyaluronic acid or pharmaceutically acceptable salts thereof in the present compositions, a concentration of between about 1 and 5 wt./wt. % of the total composition is preferred, more preferably between about 1.5 and 3.5 wt./wt. %.

### Formulation Method A

a) mixing a macromolecular pharmaceutical agent in a suitable solvent with an alkali metal alkyl sulfate, an edetate, and an alkali metal salicylate;

b) subsequently adding at least one micelle-forming compound selected from the group consisting of lecithin, hyaluronic acid, octylphenoxypolyethoxyethanol, glycolic acid, lactic acid, chamomile extract, cucumber extract, oleic acid, linoleic acid, linolenic acid, borage oil, evening of primrose oil, menthol, trihydroxy oxocholanyl glycine, glycerin, polyglyccrin, lysine, polylysine, triolein, polyoxyethylene ethers, polidocanol alkyl ethers, chenodeoxycholate, deoxycholate, pharmaceutically acceptable salts thereof, analogs thereof and mixtures or combinations thereof, to form a micellar macromolecular pharmaceutical agent composition; and,

c) after step b), adding at least one additional micelle-forming compound which is different from that added in step b) but selected from the same group. Preferably, the micelle-forming compound selected in step b) is lecithin.

Again, during or after step b), a phenolic compound as described above can be added to the composition. Mixing can be vigorous or not. Vigorous mixing may be accomplished by using high-speed stirrers, such as magnetic stirrers, propeller stirrers, or sonicators, and is preferred.

The particle size of the micelles will typically be in the range of 1 to 10 nanometers. Preferably, the micelle size ranges between 1 and 5 nanometers.

### Formulation Method B.

In a round bottom flask of 50 ml capacity, 10 mg of soluble collagen, 10 mg of elastin were weighed. The mixture was solubilized in 10 ml of sterile saline solution (0.9%). The mixture was stirred continuously. In a separate 50 ml round bottom flask, 25 mg of sphingosine and 25 mg cerebroside (or 20 mg phosphatidyl choline+25 mg phosphatidy serine) were combined and the mixture was dissolved in pure ethanol or in a mixture of 70:30 isoproanol:ethanol. The alcohol was completely removed by rotary vacuum evaporation to obtain a uniform coating of the phospholipids mixture on the flask wall. 500 mg hyaluronic acid or Na hyaluronate solution in 50 ml of (0.9%) saline was added. The flask was swirled and then stirred continuously for several minutes at room temperature.

The hyaluronic acid was thus coated uniformly with the phospholipid micelle coating. This coated and preserved micellar hyaluronic acid solution was then added to the flask containing the mixture of collagen and cross linked lowed molecular weight elastin. The solution was stirred for-about 5 minutes slowly and then kept at a room temperature in a brown glass vial.

### Formulation Method C

Ensure that all equipment is clean and sanitized and, where necessary, sanitize vessel thirty (30) minutes prior to formulation.

Phase A ingredients arc:

| | | |
|---|---|---|
| % by wt. | Ingredients | Qty. |

PHASE A

| | | |
|---|---|---|
| 0.3172 | WATER | 0.3172 |
| 0.0075 | SODIUM LAURYL SULFATE | 0.0075 |
| 0.0005 | VERSENE NA2 | 0.0005 |
| 0.001 | ALOE VERA 200 X POWDER | 0.001 |
| 0.0455 | GLYCERIN | 0.0455 |
| 0.02 | BUTYLENE GLYCOL | 0.02 |

Step 1: Add Phase A ingredients as listed into a main tank with high speed mixing and begin heating to 70-75° C. Mix for approximately thirty (30) minutes or until completely dissolved and free of any lumps.

Phase B ingredients are:

| % by wt. | Ingredients | Qty. |
|---|---|---|
| 0.11 | STEARIC ACID | 0.11 |
| 0.02 | GLYCERYL STEARATE SE | 0.02 |
| 0.0048 | CASTOR OIL | 0.0048 |
| 0.01 | OLIVE OIL | 0.01 |
| 0.004 | DIMETHICONE | 0.004 |
| 0.03 | MYRJ 52 | 0.03 |
| 0.0189 | CRODAMOL STS | 0.0189 |
| 0.0001 | SESAME OIL | 0.0001 |
| 0.005 | LECITHIN | 0.005 |
| | (SPHINGOCIDE or | |
| | (PHOSPHOLIPON G) | |

Step 2: Add Phase B ingredients as listed in a side tank with mixing and maintain a temperature of 70-75° C. Mix for approximately thirty (30) minutes or until uniform. Continue to maintain temperature at 70-75° C.

Step 3: Add mixed Phase B ingredients from Step 2 to mixed Phase A ingredients from Step 1 by mixing Phase B into Phase A main tank. Upon fully mixing the Phase B into the Phase A, commence cooling the combined mixture to 40-45° C. Cooling should occur in a controlled manner so as to cool the combined mixture in a uniform way and should occur within a period of approximately 10-30 minutes.

Phase C ingredients are:

| % by wt. | Ingredients | Qty |
|---|---|---|
| 0.0075 | RETINYL PALMITATE - (12,500 I.U./G min) | 0.0075 |
| 0.05 | LINOLEIC ACID | 0.05 |
| 0.03 | CROTEIN A-PW-(WD) | 0.03 |
| 0.001 | COLLOIDAL OATMEAL POWDER | 0.001 |
| 0.095 | CROSS LINKED-SODIUM HYALURONATE | 0.095 |
| 0.0375 | ELASTIN | 0.0375 |
| 0.0235 | MATRIXYL | 0.0235 |
| 0.0535 | MATRIXYL 3000 | 0.0535 |
| 0.005 | TOCOPHEROL ACETATE | 0.005 |
| 0.057 | LACTIC ACID 88% | 0.057 |
| 0.015 | NIACINAMIDE | 0.015 |
| 0.008 | EVENING PRIMROSE OIL | 0.008 |

Step 4: Add Phase C ingredients as listed to the main tank which has the cooled combined mixture from Step 3. Mix for approximately thirty (30) minutes or until completely uniform after the last addition.

Phase D ingredients are:

| % by wt. | Ingredients | Qty |
|---|---|---|
| 0.0075 | RITOX 35 (BRIJ 35) (LAURETH-23) | .0075 |

Step 5: Add Phase D ingredients, mix and cool mixture to 25-30° C.

Phase E ingredients arc:

| % by wt. | Ingredients | Qty |
|---|---|---|
| 0.005 | PHENOXYETHANOL | 0.005 |
| 0.005 | DIOCIDE | 0.005 |

Step 6: Add Phase E ingredients, mix and cool mixture to 25-30° C.

Phase F ingredients are:

| % by wt. | Ingredients | Qty |
|---|---|---|
| 0.005 | TEA | 0.005 |

Step 7: Add Phase F ingredients, mix and cool mixture to 25-30° C.

The resultant mixture ideally should have the following approximate characteristics:

| | |
|---|---|
| pH: | 6.0-7.0 |
| VISCOSITY: | SP # 6 @ 10 RPM: TBD |
| SP. GRAVITY: | 1.000-1.050 |
| TEMPERATURE: | 25° C. |
| COLOR: | OFF WHITE |
| ODOR: | TMS |
| APPEARANCE: | CREAM |

The following components were used to prepare a cream for topical application:

### Formulation Method D

Phase A:

| | |
|---|---|
| De-ionized Water | 74.7% |
| Tetra Sodium EDTA | 0.3-0.5% |
| Methyl Paraben | 0.2% |
| Propylene Glycol | 2.0-3.0% |
| Alpha Hydroxyacid (fruit acid) | 1.0-2.0% |
| (Total 78.2%) | |

Phase B:

| | |
|---|---|
| Cetyl Alcohol (Adol 52 NF) | 1.0-2.0% |
| Cetearyl Alcohol | 1.0-2.0% |
| Glyceryl Stearate | 1.0-2.0% |
| PEG-100 Stearate | 1-2% |
| Stearic Acid (Emersol 132) | 4.5% |
| Sorbitan Palmitate | 0.5-0.7% |
| Polysorbate-85 | 0.5-1.0% |
| HoHoba Oil | 2-5% |
| Lanolin | 1-2% |
| Tocopheryl Acetate | 0.3-0.5% |
| Dimethicone 200 | 0.5-0.7% |
| BHA | 0.1% |
| Propylparaben | 0.1% |
| Diazolidinyl UREA | 0.2% |
| (Total 13.7%) | |

Phase C:

| | |
|---|---|
| Fragrance (lilac, jasmine) | as needed |
| Aloe Vera (powder) | 0.5-1.0% |
| Retinyl A (pure) | 0.03-0.05% |
| Hyaluronic Acid (micelle) | 1.0-1.5% |
| Collagen + Elastin | 1.0-1.5% |
| Talcum Powder (Ti02) | 0.1-0.3% |
| (Total 2.63%) | |

Phase D (absorption enhancers):

| | |
|---|---|
| d-limonene | 0.7% |
| Allantoin | 0.5% |
| Fulvic Acid | 0.5% |
| Quillaja saponaria (QTS) | 0.3% |
| Myrrh Extract | 0.2% |
| Glyquin (whitening agent) | 3.5-4.0% |
| (Total 5.5%), | |

Clinical Study—Methodology and Clinical Evaluation

A Double Blind Vehicle Controlled Trial to Investigate the efficacy and tolerance of Transdermal CL1 (Restylane) versus non-CL1 (Non Crossed Linked HA) in the appearance of photodamaged skin

Topical Hyaluronic Acid (HA)—Clinical Trial 1

100 subjects 2 sites: Women 35-65 with moderate to severe photodamage: 40 CL1 (crosslinked HA—Restylane), 40 NCLI (Non crossed linked HA), 20 Vehicle 2 US sites

### Subjects and Investigators Blinded

2 week wash out, 12 week trail (evaluations 2,6 and 12 weeks), 4 week post treatment (washout)
Apply twice a day clean face

Topical Hyaluronic Acid (HA)—Clinical Trial II

Visia camera system

Objective evaluations
Goldman-Rao” photographic scale in 5 grades
Evaluation of skin roughness, skin hydration, skin radiance, smoothing effect, overall efficacy and tolerance

### Subjective Questionnaires:

### Product Qualities

Subjective improvement

Topical Hyaluronic Acid (HA)—Washout

Evaluation of sustained effect of topical HA

Patient discontinued all Topicals at day 90 and were evaluated for sustained effects at day 120.
Visia photographs
Clinical evaluations

Ninety (90) Day Result Summary I.

Blinded Investigator evaluations showed highly statistically significant improvement using the topical crosslinked HA (Restylane) over time and versus the non cross linked and vehicle in Skin Roughness, Hydration, Elasticity, Radiance, Smoothing Effect and Overall Efficacy. Most dramatic differences with Smoothing Effect and Overall Efficacy

Blinded subjective evaluations showed highly statistically significant improvement using the topical crosslinked HA (Restylane) over time and versus the non cross linked and vehicle in, Hydration, Elasticity and tightness, Texture improvement, Global Appearance Improvement and Overall Efficacy

Ninety (90) Day Result Summary I.

Overall the non crosslinked HA showed better efficiency than the vehicle but was inferior to the crosslinked HA

Wrinkle evaluation using Goldman-Rao scale was too course a measurement to show statistical differences but clinical photos showed significant improvement using the crosslinked HA

Tolerance: 97 out of 100 patients finished the trial. No dropout because of tolerance issues. No significant complaints of irritation, dryness, itching or redness. No investigator observed untoward effects. Subjects liked the product texture, color, penetration, and ease of application

Washout Results.

Blinded Investigator evaluations showed highly statistically significant continued improvement after 30 day washout using the topical crosslinked HA (Restylane) versus the non cross linked and vehicle which overall lost significant ground on improvement

Dramatic clinical improvement after washout period in categories of skin roughness, smoothing effect and overall efficiency

Discussion of results of clinical trials and evaluations.

Topical crosslinked HA (Restylane) and non crosslinked HA appears tov penetrate the skin using the unique Ionic Nano Particle Technology (“InParT”) and/or Microbead Molecule Delivery System (“MMDS”) delivery system

Topical crosslinked HA (Restylane) and to a lesser extent non crosslinkcd HA appear to have significant aesthetic enhancement effect in this double blind vehicle controlled trial in virtually every category of blinded investigator evaluations and subjective evaluations as well as in clinical photographic assessments

The benefits of the topical crosslinked HA (Restylane) continue to improve even when the product is discontinued perhaps indicating a long term benefit to the skin brought forth by collagen remodeling

Numerous modifications, variations and adaptations may be made to the particular embodiments of the invention described above, without departing from the scope of the invention, which is defined in the following claims.

What is claimed is:

1. A hyaluronic acid dispersion comprising a dispersed phase consisting of microbead particles of crosslinked hyaluronic acid or a salt thereof and a continuous phase consisting of linear hyaluronic acid.

2. The hyaluronic acid dispersion according to claim 1, wherein the fraction of dispersed phase of microbead particles of crosslinked hyaluronic acid is about 0.1 to 90% by weight, of the total hyaluronic acid dispersion.

3. The hyaluronic acid dispersion according to claim 1, wherein the particle size of the crosslinked hyaluronic acid is in an range of about 80 to 300 μm.

4. The hyaluronic acid dispersion according to claim 1, wherein the molecular weight of the crosslinked hyaluronic acid is in a range of about 80,000 to 3.7 million daltons.

5. The hyaluronic acid dispersion according to claim 1, further comprising at least one additional component selected from the group consisting of collagen, elastin, one or more absorption enhancers selected from a groups consisting of short chain alcohols, long chain alcohols, polyalcohols, amines, amides, urea, amino acids or their esters, pyrrolidones, derivatives of pyrrolidones, terpenes, derivatives of terpenes, fatty acids and their esters, macrocyclic compounds, tensides, sulfoxides, liposomes, transfersomes, lecithin vesicles, ethosomes, water surfactants polyols, and essential oils and wherein said dispersion is stabile at room temperature of between 15 and 25 degrees C. for a period of at least two (2) weeks.

6. The hyaluronic acid dispersion according to claim 1, wherein the resultant product is a clear gel.

7. The hyaluronic dispersion composition according to claim 1, wherein the composition is a preparation for cosmetic or aesthetic care selected from the group consisting of facial care, skin repair, skin enhancement, skin care, body care or orthopedic care.

8. The hyaluronic dispersion composition according to claim 1, wherein the composition is applied topically or is injected.

9. A cosmetic composition comprising a hyaluronic acid dispersion wherein a dispersed phase consists of microbead particles of crosslinked hyaluronic acid or a salt thereof and a continuous phase consists of linear hyaluronic acid.

10. The cosmetic composition according to claim 8, wherein the microbead particles of crosslinked hyaluronic acid are dispersed within the linear hyaluronic acid.
